(19) United States
(12) Patent Application Publication
(10) Pub. No.: US 2023/0243770 A1
(43) Pub. Date: Aug. 3, 2023

(54) GAS SENSOR WITH SUPERLATTICE STRUCTURE

(71) Applicant: Texas Instruments Incorporated, Dallas, TX (US)

(72) Inventors: Archana VENUGOPAL, MOUNTAIN VIEW, CA (US); Benjamin Stassen Cook, Woodinville, WA (US); Nazila Dadvand, SANTA CLARA, CA (US); Luigi Colombo, Dallas, TX (US)

(73) Assignee: Texas Instruments Incorporated, Dallas, TX (US)

(21) Appl. No.: 18/180,572

(22) Filed: Mar. 8, 2023

Related U.S. Application Data
(62) Division of application No. 16/230,045, filed on Dec. 21, 2018.
(60) Provisional application No. 62/611,554, filed on Dec. 29, 2017.

(51) Int. Cl. G01N 27/12; G01N 33/00; H01L 29/06; H01L 29/16; C23C 16/04; C23C 18/06; C23C 18/16; C23C 16/26; B33Y 10/00; B33Y 80/00

(52) U.S. Cl. CPC G01N 27/127; G01N 33/0027; G01N 27/128; H01L 29/0669; H01L 29/1606; C23C 16/042; C23C 16/04; C23C 18/06; C23C 18/1605; C23C 18/1603; C23C 16/047; C23C 18/1607; C23C 18/1657; C23C 16/26; C23C 18/1648; B33Y 10/00; B33Y 80/00; H01L 21/02606

(57) ABSTRACT

A gas sensor has a microstructure sensing element which comprises a plurality of interconnected units wherein the units are formed of connected graphene tubes. The graphene tubes may be formed by photo-initiating the polymerization of a monomer in a pattern of interconnected units to form a polymer microlattice, removing unpolymerized monomer, coating the polymer microlattice with a metal, removing the polymer microlattice to leave a metal microlattice, depositing graphitic carbon on the metal microlattice, converting the graphitic carbon to graphene, and removing the metal microlattice.

### CROSS-REFERENCE TO RELATED APPLICATIONS

This application is a Division of U.S. patent application Ser. No. 16/230,045 filed on Dec. 21, 2018 which claims priority to U.S. Provisional Patent Application No. 62/611,554 filed on Dec. 29, 2017. This application is related to U.S. patent application Ser. No. 16/229,822 filed Dec. 21, 2018, which claims priority to U.S. Provisional Patent Application No. 62/611,483 filed on Dec. 28, 2017, and to U.S. patent application Ser. No. 16/230,070 filed Dec. 21, 2018, which claims priority to U.S. Provisional Patent Application No. 62/611,499 filed on Dec. 28, 2017, and to U.S. patent application Ser. No. 16/229,827 filed Dec. 21, 2018, and to U.S. patent application Ser. No. 16/229,971 filed Dec. 21, 2018 which claims priority to U.S. Provisional Patent Application No. 62/611,511 filed on Dec. 28, 2017, the contents of which are hereby incorporated by reference in their entireties.

### FEDERALLY SPONSORED RESEARCH OR DEVELOPMENT

Not Applicable

### BACKGROUND

Graphene is a single-layer sp²-hybridized 2D network of carbon atoms that conceptually serves as the basis of many important allotropes of carbon. It can be stacked to form 3D graphite, rolled to form 1D carbon nanotubes, and fused to form 0D fullerenes. Owing to its strongly delocalized electron configuration, graphene exhibits exceptional charge carrier mobility, thermal conductivity, mechanical strength, and chemical stability. However, like other nanomaterials, the properties of graphene depend on its size, atomic structure, and physical environment. Graphene and graphene-based materials have tailorable properties that can be exploited in a broad range of devices, including transistors, capacitors, electron field emitters, transparent conductors, sensors, catalysts, and drug delivery agents. Graphene has previously been proposed for use in gas sensors—see, e.g., Novoselov et al., Nature Mat., 6, 652 (2007).

Two-dimensional (2D) sp²-bonded carbon exists in the form of graphene, buckyballs and carbon nanotubes (CNTs). Graphene is “flat” or 2D, fullerenes (“Buckyballs”) are spherical or 0D, and CNTs are tubes in 1D. Forming these materials in a singular, regular, repeatable structure has not previously been achieved. Superstructures of these materials may provide very strong, light, highly thermally and electrically conductive structures. Attempts have been made to fabricate sp²-bonded sponges as shown in FIG. 1A, however these structures are irregular and have properties that vary with position.

The isolation of graphene via the mechanical exfoliation of graphite has sparked strong interest in two-dimensional (2D) layered materials. The properties of graphene include exceptional strength, and high electrical and thermal conductivity, while being lightweight, flexible and transparent. This opens the possibility of a wide range of potential applications, including high-speed transistors and sensors, barrier materials, solar cells, batteries, and composites.

Growth of regular 3D superstructures using sp²-bonded carbon may address the shortcomings of the flexible sp² carbons for 3D applications given that hexagonally arranged carbon is strong, chemically inert, electrically and thermally conductive, and optically transparent. Such 3D superstructures may find used in a number of applications from packaging, thin optically transparent electrically conductive strong thin films, and many more.

When a carbon atom is attached to three groups (or, as in the case of graphene, three other carbon atoms) and so is involved in three a bonds, it requires three orbitals in the hybrid set. This requires that it be sp² hybridized.

An sp²-hybridized bond has 33% s and 67% p character. The three sp² hybrid orbitals point towards the corners of a triangle at 120° to each other. Each sp² hybrid is involved in a σ bond. The remaining p orbital forms the π bond. A carbon double bond may be viewed as a σ+λ bond.

### BRIEF SUMMARY

In one example, a gas sensor has as its sensing element a graphene microstructure comprised of a plurality of interconnected units wherein the units are formed of graphene tubes. The graphene tubes that form the microstructure may be arranged in an ordered structure and form symmetric patterns that repeat along the principal directions of three-dimensional space.

A method of forming such a graphene microstructure is disclosed herein which comprises: photo-initiating the polymerization of a monomer in a pattern of interconnected units to form a polymer microlattice; removing unpolymerized monomer; coating the polymer microlattice with a metal; removing the polymer microlattice to leave a metal microlattice; depositing graphitic carbon on the metal microlattice; converting the graphitic carbon to graphene; and removing the metal microlattice.

### BRIEF DESCRIPTION OF THE SEVERAL VIEWS OF THE DRAWING(S)

FIG. 1A is a schematic drawing of a fabrication process for a metal-based microlattice template in accordance with an example.

FIG. 1B is a flowchart for the fabrication process depicted schematically in FIG. 1A.

FIG. 2 is a schematic representation of a graphene-based gas sensor of the prior art.

FIG. 3 is a schematic representation of an improved version of the gas sensor depicted in FIG. 2 that has a channel for exposing both sides of the 2D graphene to gas molecules.

FIG. 4 is a schematic diagram of an exemplary gas sensor having a graphene microlattice sensing element.

### DETAILED DESCRIPTION

Although graphene is an effective gas sensor (having low density of states (DOS) and carrier concentration plus reversible chemical doping), it has certain limitations when used in the 2D form. These limitations include: substrate effects including a lack of structural stability when suspended; and, a limited surface area (a single face) available for gas detection.

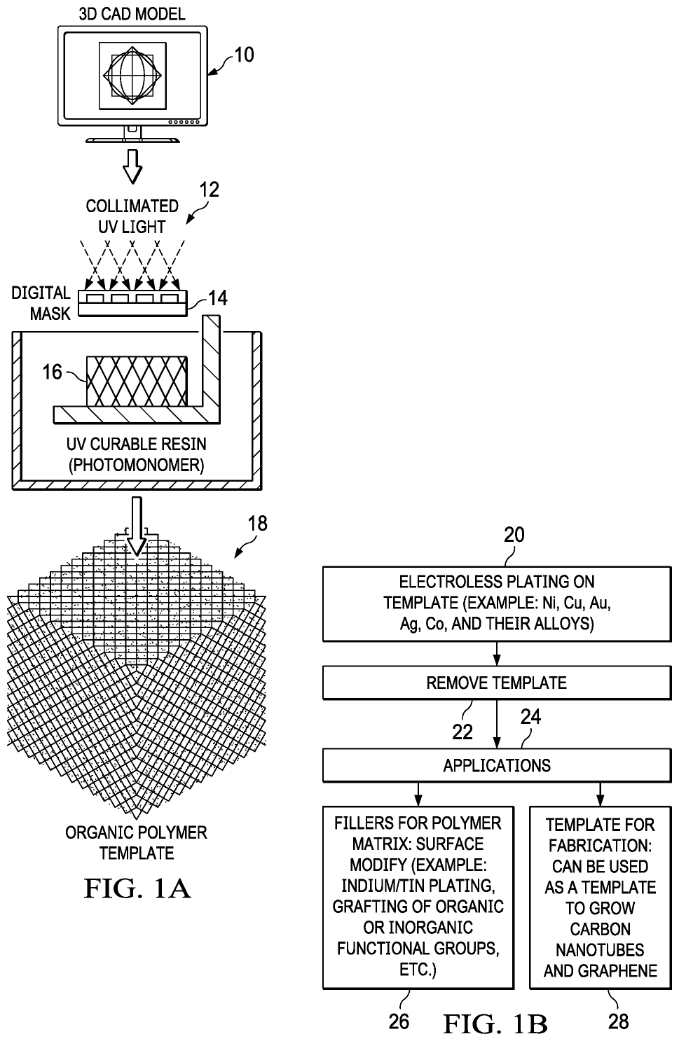

It has been found that an organic/inorganic superstructure may be used as a template for the formation of a 3D metal superstructure that may then be used to grow graphitic carbon on the surface of the metal. The template may be fabricated through a self-propagating photopolymer waveguide technique (see, e.g., Xiaoyu Zheng et. al., Ultralight, Ultrastiff Mechanical Metamaterials; Science 344 (2014) 1373-1377 and T. A. Schaedler, et al., Ultralight Metallic Microlattices; Science 334 (2011) 962-965). As illustrated schematically in FIG. 1A, an interconnected 3D photopolymer lattice may be produced upon exposure of an appropriate liquid photomonomer 16 to collimated UV light 12 through a specifically designed (e.g. using a computer-aided design model 10) digital mast 14 that contains openings with certain spacing and size. The fabricated organic polymer template microlattice 18 may then be coated by electroless copper or other suitable metal (e.g. Ni, Co, Au, Ag, Cu, and alloys thereof) followed by etching away the organic polymeric matrix (scaffold). The resulting metal-based microlattice may be then used as a template to grow the graphitic carbon. The thickness of the electroless plated metal may be controlled in the range of nanometer to micrometer by adjusting the plating time, temperature, and/or plating chemistry.

FIG. 1A schematically illustrates an exemplary fabrication process of organic polymeric microlattices (scaffolds) 18 prior to coating with electroless plating.

The present disclosure is of a gas sensor having as its sensing element a “periodically structured” carbon nanostructure. The carbon nanostructures of the prior art are irregular and have much larger dimensions than those which may be achieved using the methodology disclosed herein.

The present process may be used to create a regular array, and the superstructure dimensions (unit) and structure may be optimized for strength, thermal and other fundamental properties.

There are several aspects of this procedure that are noteworthy:

it provides a regular structure with defined dimensions;

it can form very thin metal (e.g. Ni, Co, Cu, Ag, Au) microlattices;

it enables the formation of graphitic carbon on very thin metals by a surface-limited process for very thin metal wires or tubes.

The present process uses a polymeric structure as a template for such fabrication with the subsequent formation of a metal superstructure that may then be exposed to a hydrocarbon (e.g. methane, ethylene, acetylene, benzene) to form graphitic carbon, followed by etching of the metal from under the graphitic carbon using appropriate etchants such as, for example, FeCl₃ or potassium permanganate.

Collimated UV light 12 through a photomask 14 or multi-photon lithography may be used in a photo-initiated polymerization to produce a polymer microlattice 18 comprised of a plurality of interconnected units. Exemplary polymers include polystyrene and poly(methyl methacrylate) (PMMA). Once polymerized in the desired pattern, the remaining un-polymerized monomer may be removed.

The polymer structure (polymer scaffold) 18 may then be plated with a suitable metal using an electroless plating process 20.

Electroless nickel plating (EN) is an auto-catalytic chemical technique that may be used to deposit a layer of nickel-phosphorus or nickel-boron alloy on a solid workpiece, such as metal, plastic, or ceramic. The process relies on the presence of a reducing agent, for example hydrated sodium hypophosphite (NaPO₂H₂·H₂O) which reacts with the metal ions to deposit metal. Alloys with different percentages of phosphorus, ranging from 2-5 (low phosphorus) to up to 11-14 (high phosphorus) are possible. The metallurgical properties of the alloys depend on the percentage of phosphorus.

Electroless plating has several advantages over electroplating. Free from flux-density and power supply issues, it provides an even deposit regardless of workpiece geometry, and with the proper pre-plate catalyst, may deposit on non-conductive surfaces. In contradistinction, electroplating can only be performed on electrically conductive substrates.

Before performing electroless plating, the material to be plated must be cleaned by a series of chemicals; this is known as the pre-treatment process. Failure to remove unwanted “soils” from the part's surface results in poor plating. Each pre-treatment chemical must be followed by water rinsing (normally two to three times) to remove chemicals that may adhere to the surface. De-greasing removes oils from surfaces, whereas acid cleaning removes scaling.

Activation may be done with an immersion into a sensitizer/activator solution—for example, a mixture of palladium chloride, tin chloride, and hydrochloric acid. In the case of non-metallic substrates, a proprietary solution is often used.

The pre-treatment required for the deposition of metals on a non-conductive surface usually consists of an initial surface preparation to render the substrate hydrophilic. Following this initial step, the surface may be activated by a solution of a noble metal, e.g., palladium chloride. Electroless bath formation varies with the activator. The substrate is then ready for electroless deposition.

The reaction is accomplished when hydrogen is released by a reducing agent, normally sodium hypophosphite (with the hydrogen leaving as a hydride ion) or thiourea, and oxidized, thus producing a negative charge on the surface of the part. The most common electroless plating method is electroless nickel plating, although silver, gold and copper layers can also be applied in this manner.

In principle any hydrogen-based reducing agent can be used although the redox potential of the reducing half-cell must be high enough to overcome the energy barriers inherent in liquid chemistry. Electroless nickel plating most often employs hypophosphite as the reducer while plating of other metals like silver, gold and copper typically makes use of low-molecular-weight aldehydes.

A benefit of this approach is that the technique can be used to plate diverse shapes and types of surfaces.

As illustrated in FIG. 1B, the organic polymeric microlattice may be electrolessly plated 20 with metal followed by dissolving out 22 the organic polymer scaffold. The resulting metal-based microlattice may be used in several applications 24—e.g. it may then be coated with a thin layer of immersion tin in order to prevent the metal from oxidizing during the subsequent process which may include a heat treatment. In an example, a copper/nickel super-lattice is used. The fabricated metal-based microlattice may be used as a template 28 to synthesize a graphitic carbon superstructure. The metal may then be etched out to produce a graphene microstructure comprising a plurality of interconnected units wherein the units are formed of graphene tubes that are connected by chemical electronic bonds (as distinguished from van der Waals forces which may cause carbon nanotubes to agglomerate).

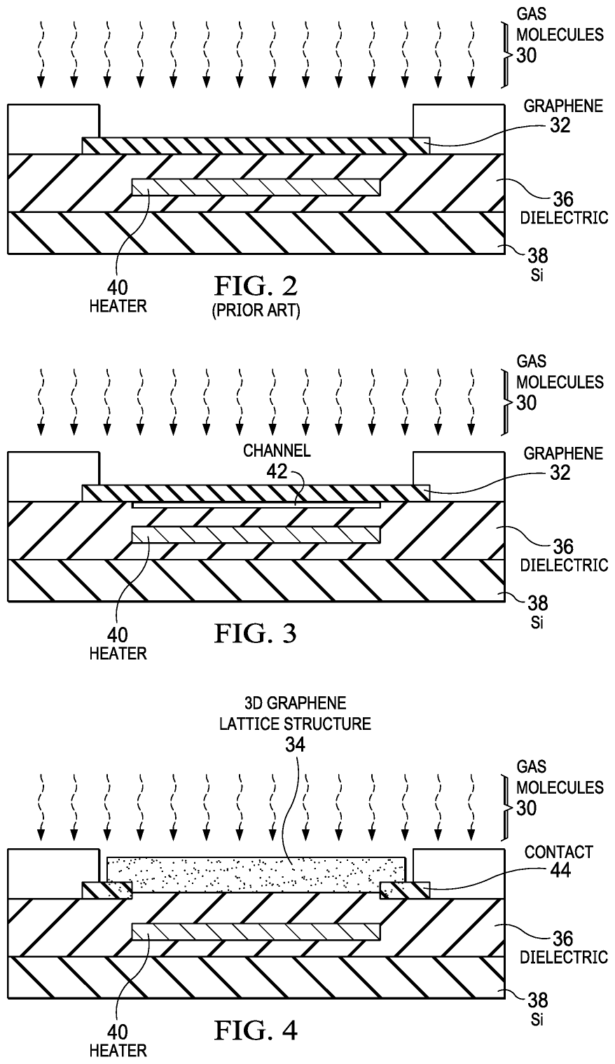

FIG. 2 shows a graphene-based gas sensor of the prior art. Graphene's electronic properties are strongly affected by the adsorption of molecules. Gas molecules 30 interact with graphene element 32 supported on dielectric 36 which is on silicon substrate 38. Heater 40 may be provided within dielectric 36 to accelerate the desorption rate of molecules adsorbed on graphene element 32.

As illustrated in FIGS. 3 and 4, a 3D graphene microstructure comprising a plurality of interconnected units wherein the units are formed of connected graphene tubes may have sufficient structural rigidity to fabricate a gas sensor having a channel 42 below the layer of graphene 32 (see FIG. 3) thereby improving the sensitivity of the gas sensor by exposing both surfaces of the graphene element to the gas molecules. Alternatively, as illustrated in FIG. 4, if the sensor element comprises a 3D graphene super-lattice structure 34 supported on dielectric-coated substrate 38 and electrically connected via contacts 44, enhanced sensitivity may obtain because of its gas permeability and increased surface area.

Modifications are possible in the described embodiments, and other embodiments are possible, within the scope of the claims.

What is claimed is:

1. A gas sensor comprising:
a silicon layer;
a dielectric layer having opposite first and second sides, the first side facing the silicon layer; and
a graphene layer on the second side of the dielectric layer, the graphene layer being exposed on at least two sides.

2. The gas sensor of claim 1, wherein the dielectric layer has cavity on the second side, and one side of the graphene layer is exposed in the cavity.

3. The gas sensor of claim 1, wherein the graphene layer includes a microstructure sensing element including a plurality of interconnected units of graphene tubes.

4. The gas sensor of claim 3, wherein the plurality of interconnected units of graphene tubes includes at least a first unit formed of first graphene tubes; and a second unit formed of second graphene tubes; and
wherein one or more of the second graphene tubes are connected to one or more of the first graphene tubes.

5. The gas sensor of claim 3, wherein the graphene tubes are arranged in an ordered structure and form symmetric patterns that repeat along principal directions of a three-dimensional space.

6. The gas sensor of claim 3, wherein the graphene tubes form a rigid structure.

7. The gas sensor of claim 3, wherein the plurality of interconnected units forms a microlattice.

8. The gas sensor of claim 3, wherein the graphene tubes are hollow.

9. The gas sensor of claim 3, wherein the graphene tubes are interconnected by chemical electronic bonds.

10. The gas sensor of claim 3, wherein the microstructure sensing element are formed by a process including:
photo-initiating polymerization of a monomer in a pattern of repeating interconnected unit cells to form a three-dimensional (3D) polymer microlattice;
removing unpolymerized monomer;
coating the 3D polymer microlattice with a metal;
removing the 3D polymer microlattice to leave a 3D metal microlattice having the pattern of repeating interconnected unit cells;
forming graphitic carbon on the 3D metal microlattice; and
removing the 3D metal microlattice to leave a 3D graphitic carbon microlattice having the pattern of repeating interconnected unit cells.

11. The gas sensor of claim 10, wherein photo-initiating the polymerization of the monomer includes at least one of: passing collimated light through a photomask, or multi-photon lithography.

12. The gas sensor of claim 10, wherein coating the polymer microlattice with a metal includes an electroless deposition of the metal.

13. The gas sensor of claim 12, wherein the electroless deposition employs hypophosphite as a reducer.

14. The gas sensor of claim 12, wherein the electroless deposition employs an aldehyde.

15. The gas sensor of claim 12, wherein the metal includes at least one of copper or nickel.

16. The gas sensor of claim 9, wherein forming the graphitic carbon on the 3D metal microlattice includes exposing the 3D metal microlattice to a hydrocarbon.
